(19) United States
(12) Patent Application Publication
(10) Pub. No.: US 2003/0199537 A1
(43) Pub. Date: Oct. 23, 2003

(54) Polymorph of a pharmaceutical

(76) Inventors: John B. Cannon, Grayslake, IL (US); Pawan Hansrani, Buffalo Grove, IL (US); Russell L. Hertzler, Libertyville, IL (US); John Lipari, Racine, WI (US); Yeshwant D. Sanzgiri, Gurnee, IL (US); Steven J. Wittenberger, Mundelein, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/125,042

(22) Filed: Apr. 18, 2002

(51) Int. Cl. A61K 31/4745; C07D 491/04

(52) U.S. Cl. 514/291; 540/456

(57) ABSTRACT

Two new crystalline polymorphs of the compound of formula (I)

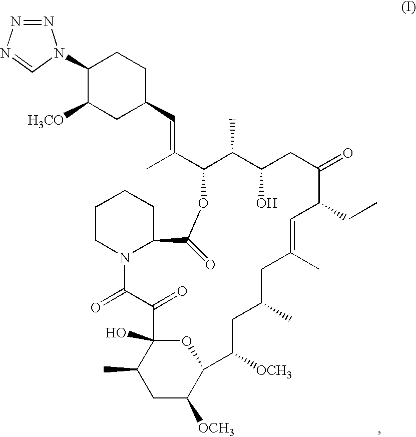

and methods for their use and preparation are disclosed.

### TECHNICAL FIELD

The present invention relates to two novel crystalline polymorphs of a known immunotherapeutic agent, methods for their preparation, methods for their interconversion, methods for their use as pharmaceutical agents, and pharmaceutical compositions comprising the novel crystalline polymorphs.

### BACKGROUND OF THE INVENTION

While the macrocycle FK-506, isolated from a strain of S. tsukuaensis, has been shown clinically to demonstrate immunosuppressive activity, its toxicity in mammals has limited its utility. The activity of FK-506 has, however, prompted efforts to discover novel analogs of FK-type compounds which possess superior properties.

One particularly potent analog is the macrocyclic lactone represented by formula (I), below. The preparation of this immunosuppressant was first disclosed in commonly owned U.S. Pat. No. 5,708,002, issued Jan. 13, 1998, which is hereby incorporated by reference in its entirety. The process disclosed in this patent produces an amorphous form of the compound.

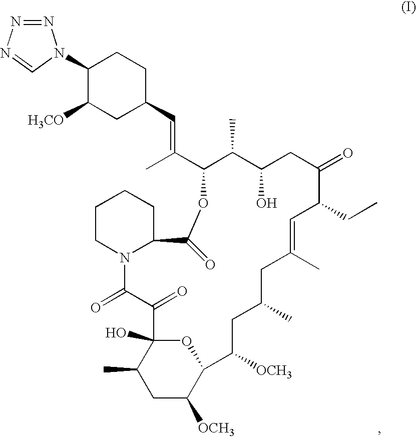

It has now been unexpectedly discovered that this macrocycle can be prepared as two novel crystalline polymorphs which are termed crystalline Forms I and II. Crystalline Form I is used in the manufacture of the compound and crystalline Form II is used in the purification of the compound in order to eliminate costly chromatography. In addition, it has been discovered that each crystalline polymorph can be made independently and that crystalline Form II can be converted to crystalline Form I.

### BRIEF DESCRIPTION OF THE DRAWINGS

FIG. 1 is a representative powder X-ray diffraction pattern of the substantially pure Form I crystalline polymorph of the compound of formula (I).

FIG. 2 is a representative powder X-ray diffraction pattern of the substantially pure Form II crystalline polymorph of the compound of formula (I).

FIG. 3 is a representative 400 MHz solid state ¹³C nuclear magnetic resonance spectrum of the substantially pure Form I crystalline polymorph of the compound of formula (I).

FIG. 4 is a representative 400 MHz solid state ¹³C nuclear magnetic resonance spectrum of the substantially pure Form II crystalline polymorph of the compound of formula (I).

FIG. 5 is a representative differential scanning calorimetric thermogram of the substantially pure Form I crystalline polymorph of the compound of formula (I).

FIG. 6 is a representative differential scanning calorimetric thermogram of the substantially pure Form II crystalline polymorph of the compound of formula (I). There are two peaks shown due to the conversion of Form II into Form I upon heating.

### SUMMARY OF THE INVENTION

In accordance with the present invention, there are two novel crystalline polymorphs of the compound of formula (I). For the sake of identification, these crystalline polymophs are designated as crystalline Form I and crystalline Form II of the compound of formula (I).

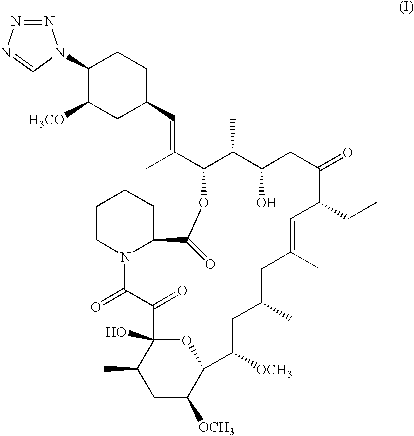

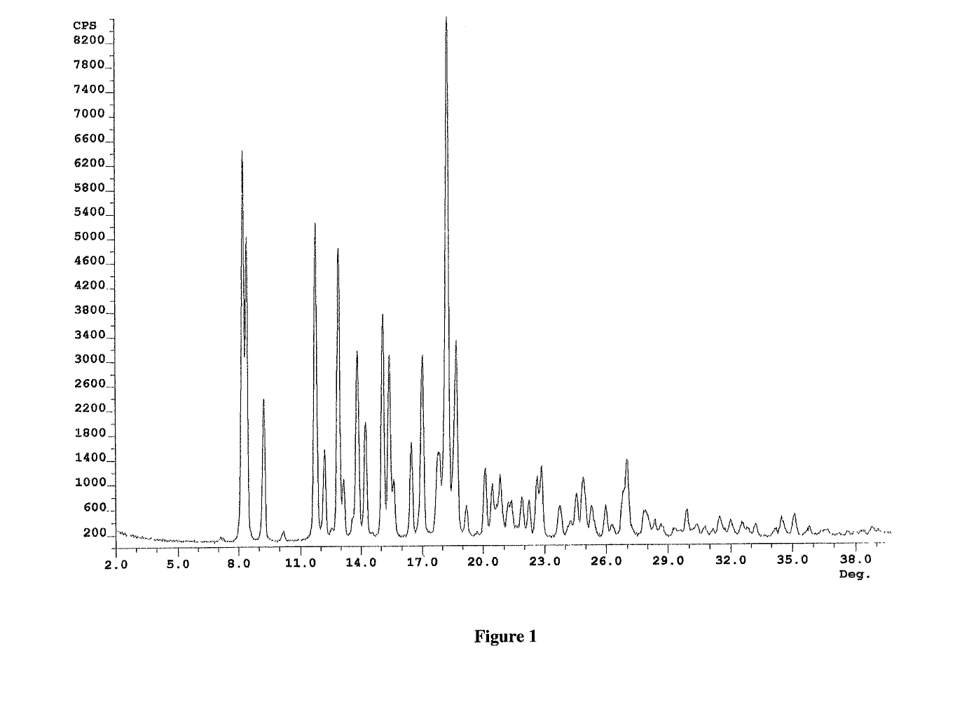

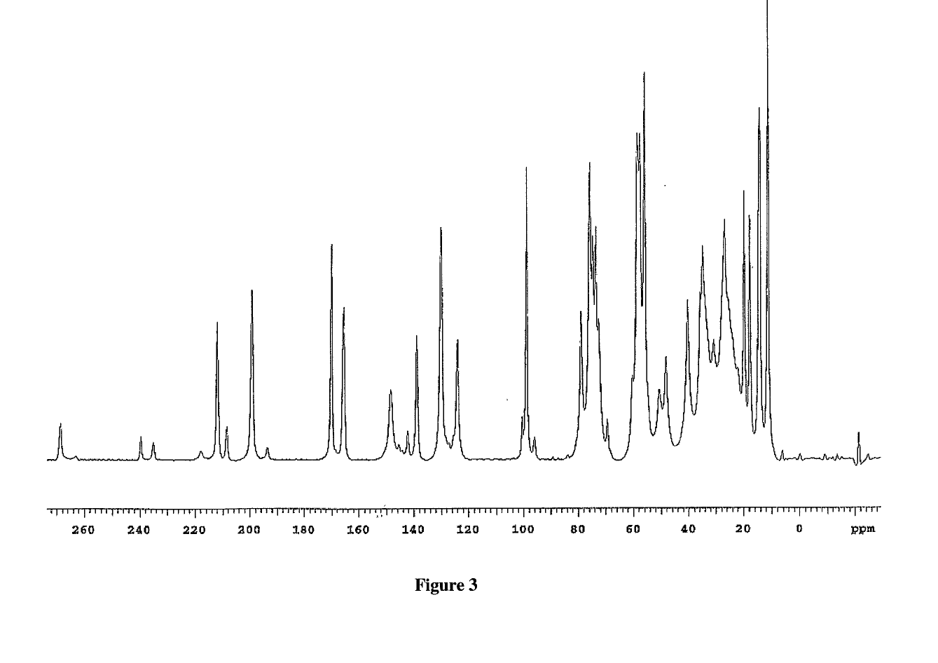

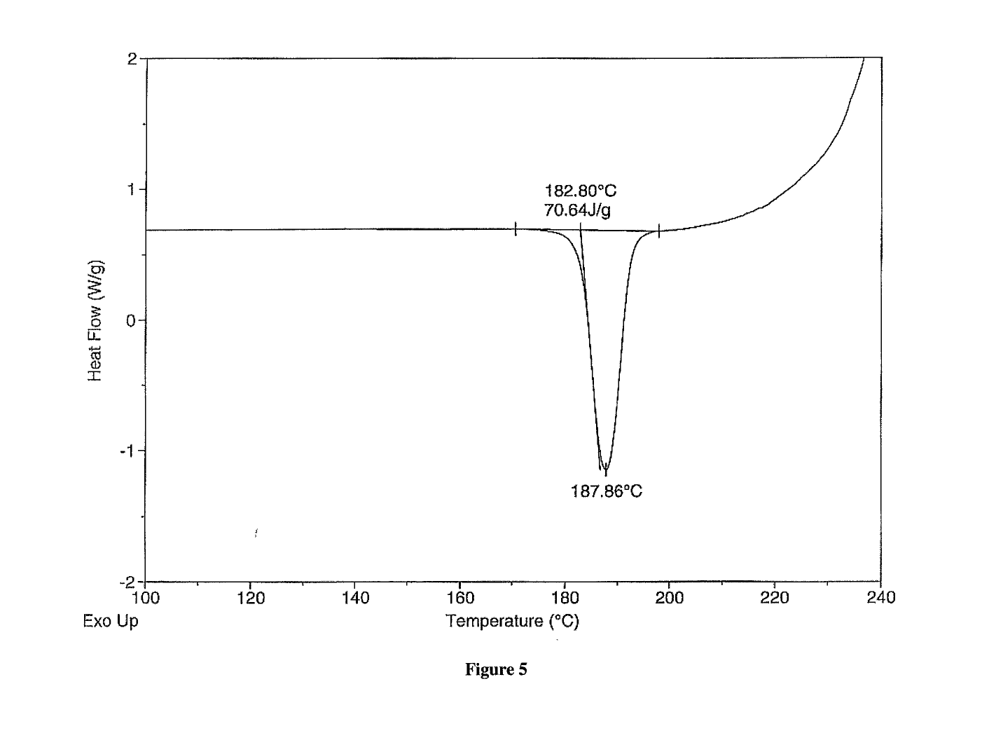

Crystalline Form I, preferably, substantially pure crystalline Form I, has the representative powder X-ray diffraction pattern, ¹³C solid state nuclear magnetic resonance spectrum, and the differential scanning calometric thermogram which appear in FIGS. 1, 3, and 5, respectively. The two-theta angle positions of characteristic peaks in the powder X-ray diffraction pattern of Form I, preferably, substantially pure Form I, as shown in FIG. 1 are 8.2°±0.1°, 8.40°±0.1°, 11.8°±0.1°, 12.9°±0.1°, 13.8°±0.1°, 15.1°±0.1°, 15.4°±0.1°, 17.0°±0.1°, 18.2°±0.1°, and 18.7°±0.1°.

Crystalline Form I, preferably, substantially pure Form I, of the compound of formula (I) can be prepared by dissolving the amorphous form of the compound of formula (I) with a suitable solvent (for example, a C₃-C₆ ester such as ethyl acetate or isopropyl acetate; most preferably isopropyl acetate), optionally filtering the solution, then treating the filtrate with an anti-solvent (for example, a C₅-C₉ alkane such as hexane or heptane; most preferably heptane) and isolating the desired polymorph. In a preferred method, the amorphous form of the compound of formula (I) is dissolved in isopropyl acetate (from about 3 L/kg to about 4 L/kg of compound, preferably 3.5 L/kg of compound) with heating (at about 70° C. to about 75° C.), and filtered. The solution is treated with heptane (from about 3.2 L/kg to about 4.3 L/kg of compound, preferably about 3.75 L/kg of compound), cooled to room temperature, stirred for about 3 hours, treated slowly with additional heptane (from about 5.0 L/kg to about 6.4 L/kg of compound, preferably about 5.7 L/kg of compound), and filtered to provide the desired polymorph.

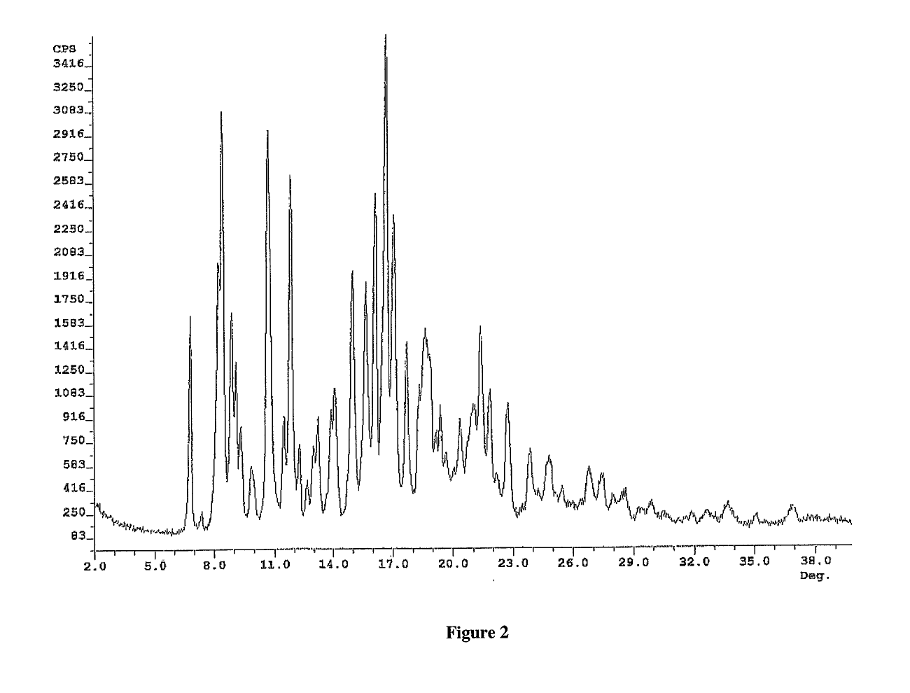

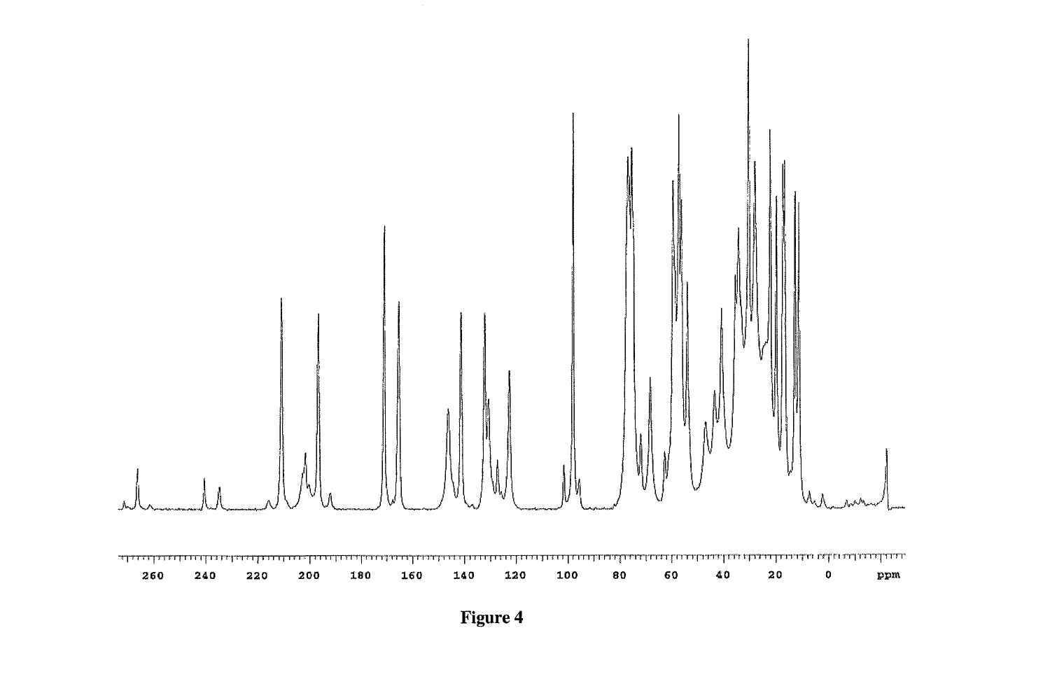

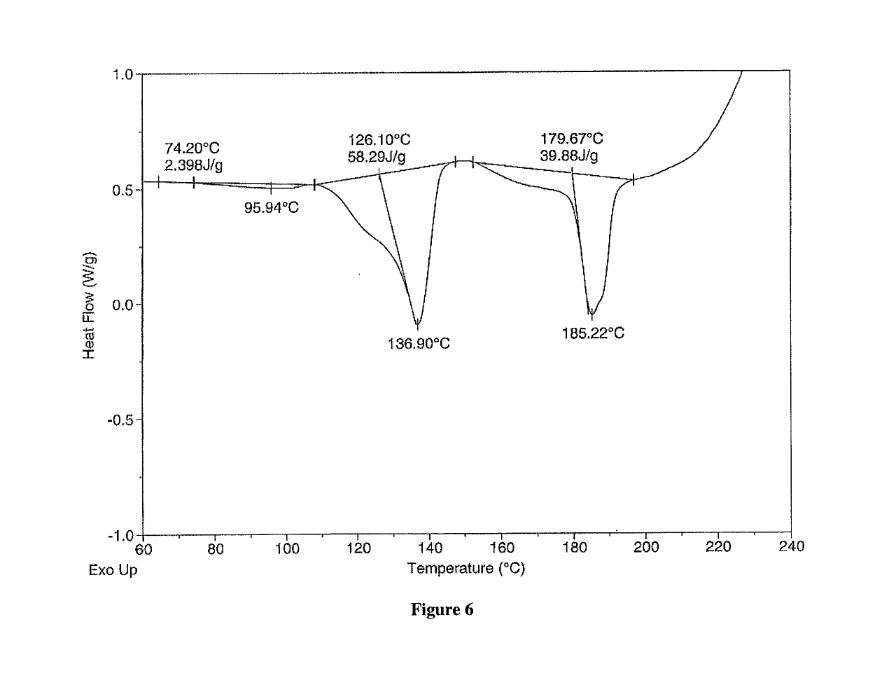

Crystalline Form II, preferably, substantially pure crystalline Form II, has the representative powder X-ray diffraction pattern, ¹³C solid state nuclear magnetic resonance spectrum, and the differential scanning calometric thermogram which appear in FIGS. 2, 4, and 6, respectively. The two-theta angle positions of characteristic peaks in the powder X-ray diffraction pattern of substantially pure crystalline Form H as shown in FIG. 2 are 6.8°±0.1°, 8.2°±0.1°, 8.4°±0.1°, 8.87°±0.10°, 10.7°±0.1°, 11.8°±0.1°, 15.0°±0.1°, 15.7°±0.1°, 16.1°±0.1°, 16.7°±0.1°, and 17.1°±0.1°.

Crystalline Form II, preferably, substantially pure crystalline Form II, of the compound of formula (I) can be prepared by dissolving the amorphous form of the compound of formula (I) in a suitable solvent (for example, a C₄-C₇ ether such as diethyl ether or methyl tert-butyl ether or a mixture of water and a C₃-C₆ ketone such as acetone or butanone; most preferably methyl tert-butyl ether or a mixture of water and 2-butanone), optionally adding a C₄-C₇ ether such as diethyl ether or methyl tert-butyl ether; most preferably methyl tert-butyl ether, and contacting the resulting mixture with Form II seed crystals. The mixture is optionally treated with an anti-solvent (for example, a C₅-C₉ alkane such as hexane or heptane; most preferably heptane), and the desired polymorph is isolated. In a preferred method, the amorphous form of the compound of formula (I) is dissolved in 93:7 wt/wt 2-butanone/water (from about 1.7 g/g of compound to about 2.1 g/g compound; preferably about 1.9 g/g of compound), heated to about 50° C., treated with methyl tert-butyl ether (from about 8.5 g/g of compound to about 10.0 g/g compound; preferably about 9.3 g/g of compound), contacted with two portions of Form II seed crystals, treated with heptane (from about 2 g/g of compound to about 9 g/g compound; preferably about 5.4 g/g of compound), cooled to room temperature, and filtered to provide the desired polymorph.

Crystalline Form I, preferably, substantially pure crystalline Form I, of the compound of formula (I) can also be prepared using crystalline Form II as an intermediate for ease of purification. Crystalline Form II is dissolved in a suitable solvent (for example, a C₃-C₆ acetate such as ethyl acetate or isopropyl acetate; most preferably isopropyl acetate) and optionally filtered. The filtrate is treated with an anti-solvent (for example, a C₅-C₉ alkane such as hexane or heptane; most preferably heptane) and then the desired polymorph is isolated by filtration. The entire process can optionally be repeated to crystalline polymorph I. In a preferred method, crystalline Form II is dissolved in isopropyl acetate (from about 2.5 mL/g of compound to about 3.5 mL/g compound; preferably about 3 mL/g of compound) and filtered. The filtrate is treated with heptane (from about 8 mL/g of compound to about 10 mL/g compound; preferably about 9.1 mL/g of compound) and the desired polymorph is isolated by filtration.

The present invention also provides a pharmaceutical composition comprising substantially pure crystalline Form I of the compound of formula (I) in combination with a pharmaceutically acceptable carrier.

The present invention also provides a pharmaceutical composition comprising substantially pure crystalline Form II of the compound of formula (I) in combination with a pharmaceutically acceptable carrier.

A preferred pharmaceutical composition for topical treatment of skin inflammation comprises a therapeutically effective amount of substantially pure crystalline Form I or Form II of the compound of formula (I) in an amount of about 1.0% by weight; 2,6-di-tert-butyl-4-methylphenol in an amount of about 0.1% by weight; isopropyl myristate in an amount of about 51.4% by weight; dimethyl isosorbide in an amount of about 9.9% by weight; transcutol in an amount of about 14.8% by weight; glycerol monostearate, self-emulsifying (1:1 glycerol monostearate/polyoxyethylene 100 stearate) in an amount of about 0.99% by weight; glycerol monolaurate in an amount of about 1.98% by weight; and ethylene vinyl acetate copolymer in an amount of about 19.8% by weight.

This pharmaceutical composition can be prepared by melting a mixture of isopropyl myristate, ethylene vinyl acetate copolymer, glycerol monostearate, self-emulsifying (1:1 glycerol monostearate/polyoxyethylene 100 stearate) and glycerol monolaurate at a temperature of about 90° C.; cooling the mixture to about 80° C.; treating the mixture with a solution of substantially pure crystalline Form I or Form II of the compound of formula (I), 2,6-di-tert-butyl-4-methylphenol, dimethyl isosorbide, and transcutol; and cooling the resulting mixture to room temperature.

The present invention also provides a method of treating a patient in need of immunosuppressant therapy comprising administering a therapeutically effective amount of substantially pure crystalline Form I of the compound of formula (I).

The present invention also provides a method of treating a patient in need of immunosuppressant therapy comprising administering a therapeutically effective amount of substantially pure crystalline Form II of the compound of formula (I).

### DETAILED DESCRIPTION OF THE INVENTION

As used herein, the singular forms “a”, “an”, and “the” include plural reference unless the context clearly dictates otherwise.

As used in the present specification the following terms have the meanings indicated:

The term “alkyl,” as used herein, refers to a monovalent group derived from a straight or branched chain saturated hydrocarbon.

The term “C₅-C₉ alkane,” as used herein, refers to a straight or branched chain hydrocarbon containing between five and nine carbon atoms. Examples of C₅-C₉ alkanes include, but are not limited to, pentane, 2,2-dimethylpentane, hexane, and nonane.

The term “anti-solvent,” as used herein, refers to a solvent which causes a compound to precipitate out of a solution.

The term “C₃-C₆ ester,” as used herein, refers to a solvent of formula RCO₂R′, containing between three and six carbon atoms, wherein R and R′ are straight or branched alkyl groups. Examples of C₃-C₆ esters include, but are not limited to, methyl acetate, ethyl acetate, and isopropyl acetate.

The term “C₄-C₇ ether,” as used herein, refers to a solvent of formula ROR′, containing between four and seven carbon atoms, wherein R and R′ are straight or branched alkyl groups. Examples of C₄-C₇ ethers include, but are not limited to, diethyl ether and methyl tert-butyl ether.

The term “C₃-C₆ ketone,” as used herein, refers to a solvent of formula RC(O)R′, containing between three and six carbon atoms, wherein R and R′ are straight or branched alkyl groups. Examples of C₃-C₆ ketones include, but are not limited to, 2-butanone, 2-hexanone, and 3-hexanone.

The term “suitable solvent,” as used herein, refers to a substance or a mixture of substances which is a liquid between about 20 and about 35° C. and is capable of being used in a recrystallization.

The term “substantially pure”, when used in reference to a polymorph of the compound of formula (I), refers to a polymorph of the compound of formula (I), crystalline Form I or crystalline Form II, which is greater than about 90% pure. This means that the polymorph of the compound of formula (I) does not contain more than about 10% of any other compound and, in particular, does not contain more than about 10% of any other form of the compound of formula (I). More preferably, the term “substantially pure” refers to a polymorph of the compound of formula (I), crystalline Form I or crystalline Form II, which is greater than about 95% pure. This means that the polymorph of the compound of formula (I) does not contain more than about 5% of any other compound and, in particular, does not contain more than about 5% of any other form of the compound of formula (I). Even more preferably, the term “substantially pure” refers to a polymorph of the compound of formula (I), crystalline Form I or crystalline Form II, which is greater than about 97% pure. This means that the polymorph of the compound of formula (I) does not contain more than about 3% of any other compound, and, in particular, does not contain more than about 3% of any other form of the compound of formula (I).

Powder X-ray diffraction analysis of the samples was conducted in the following manner: The samples for X-ray diffraction analysis were ground to a fine powder and packed into a cavity style sample holder containing a zero background plate. The samples were analyzed on a Scintag X-2 theta/theta diffractometer equipped with a normal focus copper X-ray tube operated at 1.8 kW and using a Peltier cooled detector system. Samples were scanned continuously from 2.00 to 40.00 degrees at the rate of 1 degree/ minute. The diffraction data was collected by a computer using Scintag's Diffraction Management SoftwareNT (DMSNT).

Characteristic powder X-ray diffraction pattern peak positions are reported for polymorphs in terms of the angular positions (two theta) with an allowable variability of ±0.1°. This allowable variability is specified by the U.S. Pharmacopeia, pages 1843-1884 (1995). The variability of ±0.1° is intended to be used when comparing two powder X-ray diffraction patterns. In practice, if a diffraction pattern peak from one pattern is assigned a range of angular positions (two theta) which is the measured peak position ±0.1° and if those ranges of peak positions overlap, then the two peaks are considered to have the same angular position (two theta). For example, if a diffraction pattern peak from one pattern is determined to have a peak position of 5.2°, for comparison purposes the allowable variability allows the peak to be assigned a position in the range of 5.1°-5.3°. If a comparison peak from the other diffraction pattern is determined to have a peak position of 5.3°, for comparison purposes the allowable variability allows the peak to be assigned a position in the range of 5.2°-5.4°. Because there is overlap between the two ranges of peak positions (i.e., 5.1°-5.3° and 5.2°-5.4°) the two peaks being compared are considered to have the same angular position (two theta).

Solid state nuclear magnetic resonance analysis of samples was conducted in the following manner. A Bruker AMX-400 MHz instrument was used with the following parameters: CP-MAS (cross-polarized magic angle spinning); spectrometer frequency for ¹³C was 100.627950087186 MHz; pulse sequence was vacp2lev; contact time was 3 milliseconds; temperature was ambient room temperature; spin rate was 7000 Hz; relaxation delay was 5.000 sec; 90 Deg. ¹³C pulse width was 3.6 microseconds; 90 Deg. ¹H pulse width was 4.5 microseconds; acquisition time was 0.067 seconds; sweep width was 30487.8 Hz; 4000 scans.

Differential scanning calometric analysis of the two samples was conducted in the following manner: A TA Instrument Differential Scanning Calorimeter, model 2920, and Thermal Solutions operating software, version 1.2J, was used to acquire the data. The data was analyzed using Universal Analysis software, version 2.6D. The sample weight for crystalline Form I was 4.740 mg, and the sample weight for crystalline Form II was 7.485 mg. Both samples were placed in (separate) aluminum pans which were then covered, but not sealed. Both samples were heated from room temperature to 250° C. at 10° C./min. The furnace was purged with nitrogen at 50 mL/min.

A preferred pharmaceutical composition for topical treatment of skin inflammation comprises:

(a) a therapeutically effective amount of the substantially pure crystalline Form II of the compound of formula (I) in an amount of about 1.0% by weight;

(b) 2,6-di-tert-butyl-4-methylphenol in an amount of aout 0.1%;

(c) isopropyl myristate in an amount of about 51.4% by weight;

(d) dimethyl isosorbide in an amount of about 9.9% by weight;

(e) transcutol in an amount of about 14.8% by weight;

(f) glycerol monostearate, self-emulsifying (1:1 glycerol monostearate/polyoxyethylene 100 stearate) in an amount of about 0.99% by weight;

(g) glycerol monolaurate in an amount of about 1.98% by weight; and

(h) ethylene vinyl acetate copolymer in an amount of about 19.8% by weight.

This formulation was consistently superior in the in vivo swine contact hypersensitivity efficacy testing (>50% inhibition, drug vs. placebo), showed drug penetration into human epidermis and dermis in vitro, and was physically and chemically stable (<10% degradation at 40° C., 6 months). Results of formulations containing varying percentages of the active compound are shown below.

TABLE 1
Data for Formulation R+* of Crystalline Form I of the Compound
of Formula (I) in the In vivo Swine Contact Hypersensitivity
Efficacy model

| % | Form- | Cmpd. | | | |
|---|---|---|---|---|---|
| Cmpd. | ulation | Formula | % | % Inhib. | % Inhib. |
| Formula | mg/ | (I) | Inhib. | Drug vs. | Drug vs. |
| (I) | cm² | μg/cm² | Delta† | Untreated | Placebo |
| 1 | 2 | 20 | 68 | 92 ± 2.9** | 90 ± 3.5** |
| 0.3 | 5 | 15 | 26 | 76 ± 5.1** | 60 ± 6.5** |
| 1 | 5 | 50 | 28 | 78 ± 5.4** | 59 ± 6.8** |
| 1 | 5 | 50 | 30 | 90 ± 3.2** | 79 ± 7.3** |

*Formulation abbreviations can be found in the examples listed below
†Delta = (% inhib. drug vs. untreated) − (% inhib. placebo vs. untreated)
**p < 0.05

TABLE 2
Data for Formulation R+* of Crystalline Form I of the Compound
of Formula (I) in the In vitro Human Skin Penetration

| % Cmpd. | | | |
|---|---|---|---|
| Formula | Amt. of drug | Penetration** | Penetration** |
| (I) | applied (μg) | (%) | (μg) |
| 1.0 | 82 | 1.03 | 0.92 |
| 1.0 | 292 | 0.85 | 2.55 |

*Formulation abbreviations can be found in the examples listed below
**Penetration = amount (% or μg) of drug penetrated into viable epidermis + dermis + receptor

TABLE 3
Stability Observations for Formulation R+* of Crystalline Form I
of the Compound of Formula (I)

| Physical | Chemical Stability: Potency | | | |
|---|---|---|---|---|
| Stability | % Remaining | | | |
| (40° C.) | 3 months | | 6 months | |
| † | 25° C. | 40° C. | 25° C. | 40° C. |
| Trace syneresis | 98.9% | 96.7% | 96.7% | 91.3% |

*Formulation abbreviations can be found in the examples listed below
†Physical stability: observations after 3 months at 40° C. of formulation containing 1% of the compound of formula (I)

TABLE 4
Data for Formulations of Crystalline Form I of the Compound of Formula (I)
in the In vivo Swine Contact Hypersensitivity Efficacy Model

| | % | Form- | Compound | | | |
|---|---|---|---|---|---|---|
| | Cmpd. | ulation | of | % | % Inhib. | % Inhib. |
| | Formula | mg/ | Formula | Inhib. | Drug vs. | Drug vs. |
| Formula* | (I) | cm² | (I) · g/cm² | Delta | Untreated | Placebo |
| R+ | 1 | 2 | 20 | 68 | 92 ± 2.9** | 90 ± 3.5** |
| R+ | 0.3 | 5 | 15 | 26 | 76 ± 5.1** | 60 ± 6.5** |
| R+ | 1 | 5 | 50 | 28 | 78 ± 5.4** | 59 ± 6.8** |
| R+ | 1 | 5 | 50 | 30 | 90 ± 3.2** | 79 ± 7.3** |
| L | 1 | 2 | 13.2 | 76 | 71 ± 4.7** | 74 ± 4.9** |
| L | 0.3 | 5 | 15 | 11 | 74 ± 4.6** | 39 ± 9.4** |
| L | 1 | 5 | 50 | 20 | 83 ± 4.5** | 59 ± 11.8** |
| A+ | 0.8 | 2 | 16 | 27 | 38 ± 3.8** | 31 ± 4.5** |
| A+ | 0.8 | 5 | 40 | 21 | 28 ± 7.0** | 21 ± 7.8** |
| BB+ | 3 | 2 | 60 | 26 | 42 ± 9.0** | 30 ± 11.7** |
| BB+ | 3 | 5 | 150 | 18 | 55 ± 7.8** | 26 ± 14.5 |
| U | 0.75 | 5 | 37.5 | 27 | 51 ± 4.3** | 29 ± 6.7** |

*Formulation abbreviations can be found in the examples listed below
†Delta = (% inhib. drug vs. untreated) − (% inhib. placebo vs. untreated)
**p < 0.05

TABLE 5
Data for Formulations of Crystalline Form I of Compound of
Formula (I) in the In vitro Human skin Penetration

| | % | Amt. of | | |
|---|---|---|---|---|
| | Cmpd. | Drug | | |
| | Formula | Applied | Penetration** | Penetration** |
| Formula* | (I) | (μg) | (%) | (μg) |
| R+ | 1.0 | 82 | 1.03 | 0.92 |
| R+ | 1.0 | 292 | 0.85 | 2.55 |
| L | 1.0 | 297 | 2.19 | 6.54 |
| A+ | 0.8 | 52 | 0.82 | 0.60 |
| A+ | 0.8 | 220 | 0.97 | 2.33 |
| U | 0.75 | 219 | 1.97 | 4.43 |

*Formulation abbreviations can be found in the examples listed below
**Penetration = amount (% or μg) of drug penetrated into viable epidermis + dermis + receptor

TABLE 6
Stability Observations for Formulations of Crystalline Form I
of Compound of Formula (I)

| | | Chemical Stability: Potency | | | |
|---|---|---|---|---|---|
| | Physical | % remaining | | | |
| | Stability | 3 months | | 6 months | |
| Formula* | (40° C.)† | 25° C. | 40° C. | 25° C. | 40° C. |
| R+ | Trace syneresis | 98.9% | 96.7% | 96.7% | 91.3% |
| L | Trace syneresis | n.d. | n.d. | n.d. | n.d. |
| A+ | Some syneresis | 95.0% | 82.3% | 106% | 73.8% |
| BB+ | Some syneresis | 93.5% | 100% | n.d. | n.d. |
| U | Some syneresis | n.d. | n.d. | n.d. | n.d. |

*Formulation abbreviations can be found in the examples listed below
†Physical stability: observations after 3 months at 40° C. of formulation containing 1% of the compound of formula (I)
n.d. = not determined

The following examples will serve to further illustrate the preparation of the novel crystalline polymorphs of the compound of formula (I), as well as the synthesis of crystalline Form I via crystalline Form II. Melting points are uncorrected.

### EXAMPLE 1

### Preparation of Crystalline Form I of the Compound of Formula (I)

A solution of the compound of formula (I) (1029 g, prepared according to the procedure described in U.S. Pat. No. 5,708,002, issued Jan. 13, 1998) in isopropyl acetate (3.5 L) was warmed to between 70 and 75° C. and filtered through a coarse fritted funnel. The solution was treated with heptane (3.75 L), stirred at room temperature for 3 hours, and treated with additional heptane (5.65 L) by addition funnel over approximately 3 hours. The resulting precipitate was collected by filtration and the filter cake was washed sequentially with 15% isopropyl acetate/heptane (about 2 L) and heptane (2 L). The filter cake was dried under vacuum with a nitrogen bleed at 95° C. for 60 hours to provide 837 g of crystalline Form I as a white solid. mp 180-182° C.

### EXAMPLE 2

### Preparation of Crystalline Form II of the Compound of Formula (I)

A solution of the compound of formula (I) (7.56 g, prepared according to the procedure described in U.S. Pat. No. 5,708,002, issued Jan. 13, 1998) in 93:7 wt/wt 2-butanone/water (14.2 g) was heated to about 50° C. and treated with methyl tert-butyl ether (69.9 g). The solution was treated with two portions of crystalline Form II seed crystals (about 10-15 mg total), and after about 30 minutes, the mixture was treated with heptane (41.2 g) dropwise over several minutes. The temperature was held at about 50° C. for about 1 hour, then cooled to room temperature at a rate of about 5° C./hour. The suspension was stirred at room temperature for about 4 days then filtered. The filter cake was dried under vacuum to provide 4.72 g of crystalline Form II as a whilte solid. mp 121-124° C.

### EXAMPLE 3

### Preparation of Seed Crystals of Crystalline Form II of the Compound of Formula (I)

The seed crystals can be prepared by the method described in Example 2, omitting the crystal seeding.

### EXAMPLE 4

### Purification of Crystalline Form I of the Compound of Formula (I) via Crystalline Form II

A solution of the crude compound of formula (I) (12.0 g, 3:2 ratio of N1/N2/tetrazole isomers, prepared according to the procedure described in U.S. Pat. No. 5,708,002, issued Jan. 13, 1998) in methyl tert-butyl ether (240 mL) at room temperature was stirred until the concentration in the solution stabilized to about 6 mg/mL and then filtered. The filter cake was washed with methyl tert-butyl ether and dried under vacuum to provide 4.29 g of crystalline Form II as a white solid (mp 125-131° C., 87% HPLC peak area purity). The solid was dissolved in isopropyl acetate (25 mL) with gentle warming (about 50 to about 70° C.), concentrated, and dissolved in methyl tert-butyl ether (45 mL). The suspension was warmed to gentle reflux for about 2 hours, cooled to room temperature, and stirred for about 2 days. The precipitate was collected by filtration and washed with methyl tert-butyl ether to provide 3.78 g of crystalline Form II as a solid (mp 125-127° C., 91% HPLC peak area purity). The solid was dissolved in isopropyl acetate (25 mL), concentrated, re-dissolved in isopropyl acetate (25 mL), and concentrated. The residue was dissolved in methyl tert-butyl ether (37 mL), heated to gentle reflux for about 90 minutes, cooled to room temperature, and stirred for about 20 hours. The precipitate was collected by filtration to provide 3.27 g of crystalline Form II as a solid (mp 126-129° C., 94% HPLC peak area purity). The solid was dissolved in isopropyl acetate (10 mL), and filtered through a 10-15 mM filter. The filter was washed with additional isopropyl acetate (2 mL) and the combined solutions were treated with heptane (12 mL). The mixture was stirred at room temperature for about 10 minutes, treated with additional heptane (18 mL) over 30 minutes, and stirred for about 16 hours. The precipitate was collected by filtration, rinsed with 20% isopropyl acetate/heptane, and dried under vacuum (about 0.5mm Hg) to provide 3.00 g of a solid (mp 125-130° C. and about 188° C., indicating a mixture of both crystalline Form I and crystalline Form II, HPLC peak area purity 96%). The solid was dissolved in isopropyl acetate (12 mL), heated to about 70° C., cooled to room temperature, and stirred for 1.5 hours. A sample of the solid showed no melting at 120-135° C., indicating the absence of crystalline Form II. The solid was treated with heptane (21 mL), stirred for 12-16 hours, and filtered. The filter cake was rinsed with 20% isopropyl acetate/heptane and dried under vacuum (0.05 mm Hg) to provide 2.50 g of crystalline Form I (mp 185-188° C., HPLC peak area purity 96% with 0.26% N2-tetrazole isomer, 35% recovery).

### EXAMPLE 5

### Preparation of Pharmaceutical Composition of Crystalline Form I of the Compound of Formula (I) (Polymeric Ointment Formulation “R+”)

A mixture of isopropyl myristate (51.4 g), ethylene vinyl acetate copolymer (AC400) (19.8 g, purchased from Allied Signal), glycerol monostearate, self-emulsifying (Arlacel 165) (0.99 g, purchased from Uniqema), and glyceryl monolaurate (Imwitor 312) (1.98 g, purchased from Sasol) was melted at 90° C. with stirring. The mixture was cooled to 80° C. and treated with a room temperature solution of crystalline Form I of the compound of formula (I) (1.0 g) and 2,6-di-tert-butyl-4-methylphenol (0.1 g) in diethyl isosorbide (9.9 g) and transcutol (14.8 g). The mixture was cooled to room temperature with stirring to provide the desired product.

### EXAMPLE 6

### Preparation of Pharmaceutical Composition of Crystalline Form I of the Compound of Formula (I) (Polymeric Ointment Formulation “L”)

A mixture of isopropyl myristate (29.7 g), ethylene vinyl acetate copolymer (AC400) (19.8 g, purchased from Allied Signal), and propylene glycol monolaurate (34.7 g, purchased from Gattefosse) was melted at 90° C. with stirring. The mixture was cooled to 80° C. and treated with a room temperature solution of crystalline Form I of the compound of formula (I) (1.0 g) in dimethyl isosorbide (14.9 g). The mixture was cooled to room temperature with stirring to provide the desired product.

### EXAMPLE 7

### Preparation of a Pharmaceutical Composition of Crystalline Form I of the Compound of Formula (I) (Petrolatum Ointment Formulation “A+”)

A mixture of white petrolatum USP (74.8 g), white wax (7.4 g), ceresin wax (2.0 g), and Brij 72 (5.0 g) was melted in a 60-70° C. water bath. While stirring with a homogenizer, the mixture was treated with a 60-70° C. solution of crystalline Form I of the compound of formula (I) (0.8 g) and 2,6-di-tert-butyl-4-methylphenol (0.1 g) in propylene carbonate (9.9 g). The mixture was stirred for an additional 1-2 minutes at high speed, removed from the water bath, stirred at low speed for three minutes, then stirred with a magnetic stir bar for low shear mixing and allowed to cool to room temperature to provide the desired product.

### EXAMPLE 8

### Preparation of a Pharmaceutical Composition of Crystalline Form I of the Compound of Formula (I) (Cream Formulation “BB+”)

A mixture of cetostearyl alcohol (10 g), glyceryl monostearate (10 g), glycerol monostearate, self-emulsifying (Arlacel 165, purchased form Uniqema, 2.0 g), and white wax (2.0 g) was melted at 50-60° C. with stirring. While mixing with a homogenizer, water (42 g) (heated to 50° C.) was added. The mixture was treated with a 50-60° C. suspension of crystalline Form I of the compound of formula (I) (3.0 g) in dimethyl isosorbide (10 g), transcutol (10 g), propylene carbonate (10 g), and 2,6-di-tert-butyl-4-methylphenol (0.1 g). Germaben (1.0 g) was added and the mixture was cooled to room temperature with stirring to provide the desired product.

### EXAMPLE 9

### Preparation of an Alternative Cream Pharmaceutical Composition of Crystalline Form I of the Compound of Formula (I) (Alternative Cream Formulation “U”)

The crystalline Form I of the compound of formula (I) (0.75 g) was dissolved in Capmul MCM (58 g, purchased from Abitec) at 65° C. with stirring. White wax (11.6 g) was added and melted while mixing with a Motomatic stirrer. A dispersion of Carbopol 980 (0.6 g, purchased from B.F. Goodrich) in water (29 g) was added, followed by triethanolamine (0.1 g) while mixing thoroughly to neutralize and gel the Carbopol. The product was mixed with the Biospec homogenizer for 1-2 minutes and allowed to come to room temperature.

The foregoing is merely illustrative of the invention and is not intended to limit the invention to the disclosed embodiments. Variations and changes which are obvious to one skilled in the art are intended to be within the scope and nature of the invention which are defined in the appended claims.

What is claimed is

1. The crystalline polymorph of the compound of formula (I) 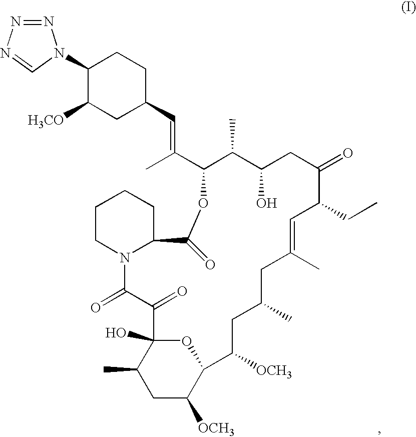
with characteristic peaks in the powder X-ray diffraction pattern at values of two theta of 8.2°±0.1°, 8.4°±0.1°, 11.8°±0.1°, 12.9°±0.1°, 13.8°±0.1°, 15.1°±0.1°, 15.4°±0.1°, 17.0°±0.1°, 18.2°±0.1°, and 18.7°±0.1°.

2. The substantially pure crystalline polymorph of the compound of formula (I) with characteristic peaks in the powder X-ray diffraction pattern at values of two theta of 8.2°±0.1°, 8.4°±0.1°, 11.8°±0.1°, 12.9°±0.1°, 13.8°±0.1°, 15.1°±0.1°, 15.4°±0.1°, 17.0°±0.1°, 18.2°±0.1°, and 18.7°±0.1°.

3. The crystalline polymorph of the compound of formula (I) with characteristic peaks in the powder X-ray diffraction pattern at values of two theta of 6.8°±0.1°, 8.2°±0.1°, 8.4°±0.1°, 8.87°±0.1°, 10.7°±0.1°, 11.8°±0.1°, 15.0°±0.1°, 15.7°±0.1°, 16.1°±0.1°, 16.7°±0.1°, and 17.1°±0.1°.

4. The substantially pure crystalline polymorph of the compound of formula (I) with characteristic peaks in the powder X-ray diffraction pattern at values of two theta of 6.8°±0.1°, 8.2°±0.1°, 8.4°±0.1°, 8.87°±0.1°, 10.7°±0.1°, 11.8°±0.1°, 15.0°±0.1°, 15.7°±0.1°, 16.1°±0.1°, 16.7°±0.1°, and 17.1°±0.1°.

5. A process for the preparation of the substantially pure crystalline polymorph of claim 2 comprising:
(a) dissolving the compound of formula (I) in a suitable solvent;
(b) optionally filtering the product of step (a);
(c) treating the filtrate of step (b) with an anti-solvent; and
(d) isolating the desired polymorph thus obtained.

6. The process of claim 5 wherein the suitable solvent is a C₃-C₆ ester.

7. The process of claim 6 wherein the suitable solvent is isopropyl acetate.

8. The process of claim 5 wherein the anti-solvent is a C₅-C₉ alkane.

9. The process of claim 8 wherein anti-solvent is heptane.

10. The process of claim 5 wherein step (a) is conducted at about 70 to about 75° C.

11. The process of claim 5 wherein step (c) is conducted at about 20 to about 25° C.

12. A process for the preparation of the substantially pure crystalline polymorph of claim 4 comprising:
(a) dissolving the compound of formula (I) with a suitable solvent;
(b) optionally treating the product of step (a) with a C₄-C₇ ether; (c) optionally treating the product of step (b) with an anti-solvent;
(d) stirring the product of step (c); and
(e) isolating the desired polymorph thus obtained.

13. The process of claim 12 wherein crystalline Form II of the compound of formula (I) is used to induce crystallization.

14. The process of claim 12 wherein suitable solvent is a C₄-C₇ ether.

15. The process of claim 14 wherein the suitable solvent is methyl tert-butyl ether.

16. The process of claim 12 wherein the suitable solvent is a mixture of a C₃-C₆ ketone and water.

17. The process of claim 16 wherein the suitable solvent is a mixture of 2-butanone and water.

18. The process of claim 18 wherein the C₄-C₇ ether is methyl tert-butyl ether.

20. The process of claim 12 wherein the anti-solvent is a C₅-C₉ alkane.

21. The process of claim 20 wherein the anti-solvent is heptane.

22. The process of claim 12 wherein steps (a), (b), and (c) are conducted at about 50° C.

23. The process of claim 12 wherein step (d) is conducted at about 20 to about 25° C.

24. A process for the preparation of the substantially pure crystalline polymorph of claim 2 comprising:
(a) dissolving the substantially pure crystalline polymoph of claim 4 in a suitable solvent;
(b) optionally filtering the product of step (a);
(c) treating the filtrate of step (b) with an anti-solvent; and
(d) isolating the desired polymorph thus obtained.

25. The process of claim 24 wherein the suitable solvent is a C₃-C₆ ester.

26. The process of claim 25 wherein the suitable solvent is isopropyl acetate.

27. The process of claim 24 wherein the anti-solvent is a C₅-C₉ alkane.

28. The process of claim 27 wherein the anti-solvent is heptane.

29. The process of claim 24 wherein step (a) is conducted at about 70 ° C.

30. The process of claim 24 wherein step (c) is conducted at about 25 ° C.

31. A pharmaceutical composition comprising the substantially pure crystalline polymorph of claim 2 in combination with a pharmaceutically acceptable carrier.

32. A pharmaceutical composition comprising the substantially pure crystalline polymorph of claim 4 in combination with a pharmaceutically acceptable carrier.

33. A pharmaceutical composition for topical treatment of skin inflammation comprising:
(a) a therapeutically effective amount of the substantially pure crystalline polymorph of claim 2 in an amount of about 1.0% by weight;
(b) 2,6-di-tert-butyl-4-methylphenol in an amount of about 0.1%;
(c) isopropyl myristate in an amount of about 51.4% by weight;
(d) dimethyl isosorbide in an amount of about 9.9% by weight;
(e) transcutol in an amount of about 14.8% by weight;
(f) glycerol monostearate, self-emulsifying (1:1 glycerol monostearate/polyoxyethylene 100 stearate) in an amount of about 0.99% by weight;
(g) glycerol monolaurate in an amount of about 1.98% by weight; and
(h) ethylene vinyl acetate copolymer in an amount of about 19.8% by weight.

34. A method for preparing a pharmaceutical composition for topical treatment of skin inflammation comprising:
(a) melting a mixture of isopropyl myristate, ethylene vinyl acetate copolymer, glycerol monostearate, self-emulsifying (1:1 glycerol monostearate/polyoxyethylene 100 stearate), and glycerol monolaurate at a temperature of about 90° C.;
(b) cooling the product of step (a) to about 80° C.;
(c) treating the product of step (b) with a solution of the substantially pure crystalline polymorph of claim 2 and 2,6-di-tert-butyl-4-methylphenol in dimethyl isosorbide, and transcutol; and
(d) cooling the product of step (c) to room temperature.

35. A method of treating a patient in need of immunosuppressant therapy comprising administering a therapeutically effective amount of the substantially pure crystalline polymorph of claim 2.

36. A method of treating a patient in need of immunosuppressant therapy comprising administering a therapeutically effective amount of the substantially pure crystalline polymorph of claim 4.
